(19) United States
(12) Patent Application Publication
(10) Pub. No.: US 2005/0251178 A1
(43) Pub. Date: Nov. 10, 2005

(54) Hooked rod delivery system for use in minimally invasive surgery

(76) Inventors: Michael V. Tirabassi, Sturbridge, MA (US); Kevin P. Moriarty, Wilbraham, MA (US); Richard B. Wait, Longmeadow, MA (US)

Correspondence Address: HOLLAND & BONZAGNI, P.C., 171 DWIGHT ROAD, SUITE 302, LONGMEADOW, MA, 01106-1700, US

(21) Appl. No.: 11/119,649

(22) Filed: May 2, 2005

Related U.S. Application Data
(60) Provisional application No. 60/567,538, filed on May 4, 2004.

(51) Int. Cl. A61B 17/08

(52) U.S. Cl. 606/153

(57) ABSTRACT

A delivery system for use in minimally invasive surgery that is made up of a substantially cylindrical hollow sleeve and a hooked deployment rod adapted to slide within the hollow sleeve is provided. The inventive delivery system serves to protect and pass anastomotic devices such as self-closing surgical devices (e.g., self-closing surgical clip assemblies) through laparoscopic and thoracoscopic parts. The subject invention also provides a method for protecting and passing such anastomotic devices through laparoscopic and thoracoscopic parts and for precisely delivering these devices to operating or surgical sites.

### RELATED APPLICATION

This application claims priority from U.S. Provisional Patent Application Ser. No. 60/567,538, filed May 3, 2004.

### TECHNICAL FIELD OF THE INVENTION

The present invention basically relates to a delivery system for use in minimally invasive surgery, and more particularly relates to a hooked rod delivery system that serves to protect and pass anastomotic devices such as self-closing surgical devices (e.g., self-closing surgical clip assemblies) through laparoscopic and thoracoscopic parts.

### BACKGROUND OF THE INVENTION

Endoscopic surgical procedures are regularly used to evaluate and treat conditions of the airway (laryngoscopy, bronchoscopy), the chest (thoracoscopy), abdomen (laparoscopy) and urinary tract (cystoscopy). Each step of any one of these procedures is performed by means of elongated instruments that are inserted into a body cavity via trocars. The benefits attributed to these minimally invasive surgical procedures include reduced patient trauma, reduced risk of post-operative infection and reduced recovery times.

Types of elongated instruments used in laparoscopic and thoracoscopic procedures include elongated instruments for delivering needles with surgical thread, clamps, clips, or fasteners.

By way of example, U.S. Pat. No. 6,197,035 B1 to Loubens et al. relates to an applicator intended for use with endoscopic procedures, which serves to set a needle secured to its suture thread in place in a surgical site. The applicator basically comprises: (1) an elongated sleeve 5 comprising a bore 6 having a surgical needle 2 disposed within the bore 6 near its second end; (2) a rod 13 with flat or planar ends that is adapted to slide within the bore 6 and rest adjacent needle 2; and (3) a retaining mechanism 17 for maintaining the position of the rod 13 with respect to the elongated sleeve 5. In one embodiment, as shown in FIGS. 7 and 8 of this reference, the rod 13 has a hook-shaped notch 26 located near one end.

In operation, the needle 2 with suture thread 9 is introduced into the bore 6 of sleeve 5. The rod 13 is then introduced into the bore 6 such that the end of rod 13 is in contact with the needle 2. The applicator is then arranged inside a trocar 3 and the rod 13 pushed down to deposit the needle in the surgical site. Once suturing is completed, the hook-shaped notch 26 is used to recover the suture thread 9 and to lead it out of the body, leaving the needle 2 inside so that extra-corporal knots can be tied. See Col. 4, lines 21 to 27, of U.S. Pat. No. 6,197,035 B1.

The applicator disclosed in this reference requires movement of the needle 2 (with attached suture thread 9) along the length of sleeve 5 toward free end 16, thereby increasing the possibility that needle 2 could be damaged, or detached from its suture thread 9 during this process. In addition, the position of hook-shaped notch 26 on rod 13 renders notch 26 difficult to use for anything other than recovering suture thread 9 and leading it out of the body.

It is further noted that the applicator disclosed in U.S. Pat. No. 6,197,035 B1, as well as other known instruments for delivering needles, clamps, clips, and the like through laparoscopic and thoracoscopic parts, have not been promoted nor used to deliver self-closing surgical devices in un-released or un-fired states to operating or surgical sites.

Self-closing surgical devices and in particular U-CLIP™ anastomotic devices have become increasingly popular as a technology that facilitates interrupted anastomotic techniques. Anastomoses with interrupted sutures are preferred in a number of different procedures. In specific regard to vascular anastomoses, interrupted sutures are preferred in procedures involving children because of the growth of vascular structures and in cases in which low-pressure vessels are involved. In the past, vascular anastomoses with interrupted sutures were hand-sewn. Because this process was very time consuming, requiring considerable manipulation of the vessels and extensive knot tying, many surgeons opted for running sutures.

The U-CLIP™ anastomotic device was designed by Coalescent Surgical, Inc. of Sunnyvale, Calif. The device consists of a self-closing surgical clip attached to a conventional surgical needle via a flexible member. The clip is reportedly fabricated from nitinol which has the property of shape memory, allowing closure of the arms upon activation of a simple release mechanism. The conventional surgical needle allows the surgeon to use conventional needle drivers and preserves the ability to align tissue precisely. The self-closing feature eliminates knot tying and suture management. As such, the interrupted anastomotic technique is enabled by this technology.

Unfortunately, delivering “shape memory” devices by way of laparoscopic or thoracoscopic means has been deemed undesirable where short or long periods of compression of these devices within an introduction sleeve could lead to premature misfires of the device upon release from the sleeve. More specifically, due to its self-closing feature, U-CLIP™ anastomotic devices have been deemed vulnerable to premature closing if compressed and then subjected to a sudden release of compressive force.

In view of the above, it is a stated object of the present invention to not only address the deficiencies of prior art laparoscopic and thoracoscopic delivery means but also to dispel preconceived notions regarding the viability of delivering “shape memory” devices through laparoscopic and thoracoscopic parts.

It is a more particular object of the present invention to provide a relatively simple delivery system that serves to protect and pass anastomotic devices (including self-closing surgical clip assemblies) through laparoscopic and thoracoscopic parts and to precisely deliver these devices to operating or surgical sites.

It is a further object of the present invention to provide a method for protecting and passing anastomotic devices through parts used in minimally invasive surgery and for precisely delivering these devices to operating or surgical sites.

### SUMMARY

The present invention therefore provides a hooked rod delivery system, which accomplishes the objects or goals identified above, and which basically comprises: a substantially cylindrical hollow sleeve; and a hooked deployment rod adapted to slide within the hollow sleeve.

The present invention further provides a method for protecting and passing anastomotic devices such as self-closing surgical devices (e.g., self-closing surgical clip assemblies) through laparoscopic and thoracoscopic parts and for precisely delivering such devices to operating or surgical sites, said method comprising:
(a) providing a hooked rod delivery system having a distal end and a proximal end, which comprises: a substantially cylindrical hollow sleeve; and a hooked deployment rod slidably arranged within the hollow sleeve, wherein the hooked deployment rod has a distal end and a proximal end, and wherein the distal end of the hooked deployment rod has a hook-shaped member either formed therein or attached thereto;
(b) causing the distal end of the deployment rod with hook-shaped member to emerge from the hollow sleeve;
(c) engaging an anastomotic device with the hook-shaped member of the deployment rod;
(d) withdrawing the distal end of the deployment rod with engaged anastomotic device into the hollow sleeve of the hooked rod delivery system, thereby compressing the anastomotic device;
(e) positioning the hooked rod delivery system at or near an operating or surgical site by inserting the distal end of the delivery system through laparoscopic or thoracoscopic parts;
(f) moving the deployment rod down toward the operating or surgical site, thereby causing the distal end of the deployment rod with engaged anastomotic device to emerge from the hollow sleeve; and
(g) rotating the deployment rod until the anastomotic device is released or disengaged from the hook-shaped member.

Other features and advantages of the invention will be apparent to one of ordinary skill from the following detailed description and accompanying drawings.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

Particular features of the disclosed invention are illustrated by reference to the accompanying drawings in which:

FIG. 1 is a partial side view of the hooked rod delivery system of the present invention;

FIG. 2 is a partial side view of the inventive delivery system of FIG. 1 showing the hooked deployment rod grasping or engaging a self-closing surgical clip with attached surgical needle;

FIG. 3 is a partial side view of the inventive delivery system of FIG. 2 after the deployment rod with engaged clip and surgical needle has been retracted into the sleeve;

FIG. 4 is an enlarged, perspective view of a released or fired self-closing surgical clip; and

FIG. 5 is a partial side view of an operating or surgical site showing a plurality of self-closing surgical clips released or fired around joined vessel walls.

### BEST MODE FOR CARRYING OUT THE INVENTION

By way of the subject invention, the present inventors have made the surprising discovery that “shape memory” devices such as self-closing surgical clip assemblies may be successfully delivered to operating or surgical sites in a minimally invasive way using a relatively simple delivery system.

Referring now to the drawings in detail, the delivery system of the present invention is shown and generally designated by reference numeral 10.

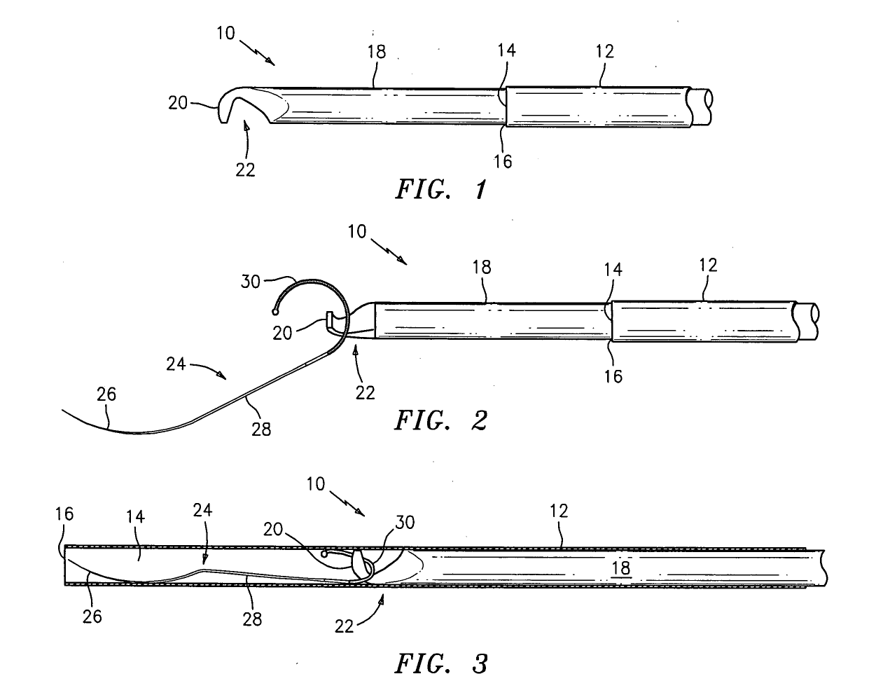

As best shown in FIG. 1, the inventive delivery system basically comprises: a substantially cylindrical hollow sleeve 12 defining a bore 14 and having a distal end 16 and a proximal end (not shown); and a hooked deployment rod 18 adapted to slide within the bore 14 of sleeve 12 and having a distal end 20 and a proximal end (not shown).

The cylindrical sleeve 12, which is adapted to slide over deployment rod 18, has an outside diameter that is set so as to facilitate insertion of the sleeve 12 through a laparoscopic or thoracoscopic part (e.g., a trocar or trocar-tube) and may be made of any suitable medical grade material, such as a medical grade metal (e.g., brass, stainless steel, titanium) or polymer (e.g., polycarbonate). In a preferred embodiment, cylindrical sleeve 12 is fabricated from a substantially transparent polymeric material, such as polycarbonate.

A scale or series of gradation marks may be placed on the outer surface of cylindrical sleeve 12 to assist a surgeon in estimating the amount of suture thread to be delivered to an operating or surgical site.

In addition, the proximal end of the cylindrical sleeve 12 may be equipped with a handle or grip to aid in the handling of system 10. Handles similar to those used with control syringes are preferred, where such handles facilitate single handed usage.

The deployment rod 18 employs a hook-shaped member 22 to facilitate grasping or engaging an anastomotic device. In a preferred embodiment, the member 22 is formed in the thickness of deployment rod 18 and extends to or beyond the central or longitudinal axis of rod 18. The exact shape or configuration of the hook-shaped member is not limited.

As will be readily apparent to those skilled in the art, the hook-shaped member 22 of deployment rod 18 facilitates not only the delivery of anastomotic devices to operating or surgical sites, but also the recovery or removal of, for example, needles and suture threads, from the site upon completion of a procedure.

Hooked deployment rod 18, which is adapted to slide within sleeve 12, has an outside diameter that closely approximates the inside diameter of sleeve 12 and may be made of any suitable medical grade material, such as the medical grade metals and polymers referenced above.

The proximal end of deployment rod 18 may also be equipped with a handle or grip to facilitate handling.

The delivery system 10 of the present invention may be provided with means for holding or retaining deployment rod 18 within sleeve 12 in either an extended or retracted position. Holding means may comprise mechanical and/or magnetic holding means, with suitable mechanical holding means including, but not limited to, grooves and matching protrusions or lugs (either friction or spring-loaded) that may be formed on the exterior of the deployment rod 18 and on the interior of the cylindrical sleeve 12, spiral channels and matching detents (either friction or spring-loaded) that may be formed on the exterior of the rod 18 and on the interior of the sleeve 12, spring loaded balls fitted with grooves, and other suitable friction or spring-loaded type devices. Suitable magnetic holding means include, but are not limited to, permamagnet and electromagnet type devices, with the electromagnet type devices either permanently activated or activated remotely by electrical signals.

The inventive delivery system 10 may be used to safely and precisely deliver anastomotic devices such as self-closing surgical devices (e.g., self-closing surgical clip assemblies), suture thread, and needles secured to suture thread to surgical sites.

In operation, an anastomotic device would be pulled into and held in place within the cylindrical sleeve 12 by the hooked deployment rod 18. The resulting assembly would then be arranged inside a trocar or trocar tube and the hooked deployment rod 18 pushed down to deposit the anastomotic device in the surgical site. Where the inventive delivery system 10 does not require movement of the anastomotic device along the entire length of sleeve 12, the risk of damage to, or premature deployment of, the anastomotic device is reduced.

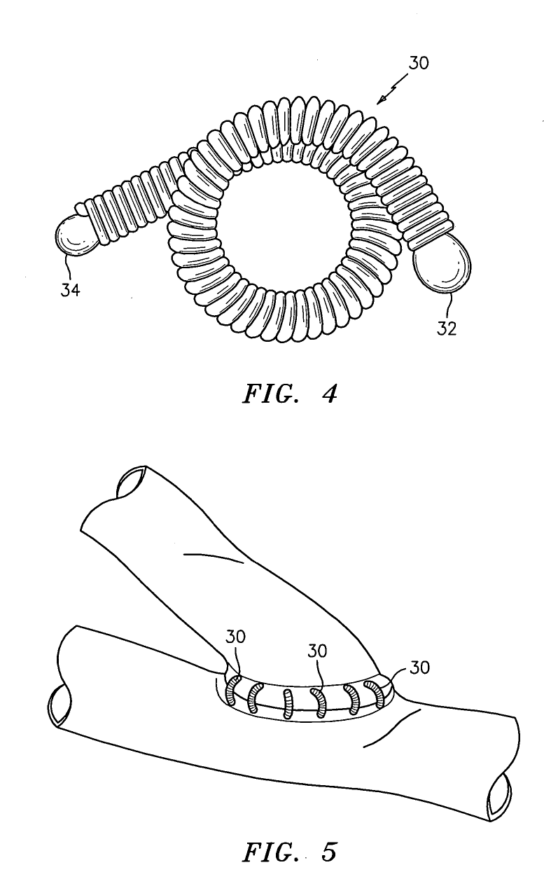

Use of the inventive hooked rod delivery system 10 with a self-closing surgical clip assembly such as the U-CLIP™ anastomotic device is shown in FIGS. 2, 3 and 5. As alluded to in the background section, a U-CLIP™ anastomotic device is sold as a clip assembly 24 that comprises a needle 26, a flexible thread-like member 28, and a self-closing surgical clip 30 which, as best shown in FIG. 4, comprises a retainer end 32 and a release mechanism end 34.

Prior to delivering the clip assembly 24 to a surgical site, the needle 26 is preferably straightened to a ski shape for ease of use and for reducing compression stress. A surgeon would then push on the deployment rod 18 of delivery system 10 in order to cause the distal end 20 to emerge from the sleeve 12. As shown in FIGS. 2 and 3, the hook-shaped member 22 on the distal end 20 of rod 18 would then be used to grasp or engage the clip 30 and the rod 18 would then be pulled back into sleeve 12.

The surgeon would then introduce the delivery system 10 into the operating or surgical site by way of a trocar or trocar tube (not shown) and push on the deployment rod 18 in order to cause clip assembly 24 to emerge from the sleeve 12. The rod 18 would then be rotated until the assembly 24 was released or disengaged from the hook-shaped member 22. The surgeon would grasp the needle 26 using e.g. laparoscopic needle drivers and then make his/her suture point by using the needle 26 to penetrate into and through the tissues to be secured together. As the needle 26 is withdrawn from the joined tissues, the flexible member 28 and clip 30 would be drawn through the tissues until the retainer end 32 of clip 30 contacts an outer tissue surface. At that time, the surgeon would use another instrument to squeeze the release mechanism end 34 of clip 30 thereby causing the needle 26 and flexible member 28 to separate from the clip 30 and, as best shown in FIG. 5, simultaneously causing the clip 30 to release or fire (i.e., close around the joined tissues).

To recover the needle 26 and attached flexible member 28, the surgeon would push on the deployment rod 18 in order to cause the distal end 20 to emerge from the sleeve 12. The surgeon would then use the hook-shaped member 22 on the distal end 20 of rod 18 to grasp or engage the flexible member 28 and would then pull the rod 18 back into sleeve 12 and withdraw the delivery system from the surgical site.

The method and delivery system 10 of the present invention were tested and evaluated by first passing U-CLIP™ anastomotic devices (having clip diameters of 1.25 millimeters (mm) and 1.50 mm when released or fired) into and out of the hollow sleeve 12 of delivery system 10. No misfires of the anastomotic device occurred even after remaining in the sleeve 12 for greater than 10 minutes.

The inventive delivery system 10 was also used to deliver U-CLIP™ anastomotic devices (also having clip diameters of 1.25 mm and 1.50 mm when released or fired) for thoracoscopic esophageal reconstruction in four piglets, each weighing approximately 4 kilograms. There was one non-survival training piglet, and three piglets that survived more than 2 months. All of the U-CLIP™ anastomotic devices functioned properly in the esophageal anastamoses after being transported through 3 millimeter ports.

Although the present invention has been shown and described with respect to detailed embodiments thereof, it will be understood by those skilled in the art that various changes in form and detail thereof may be made without departing from the spirit and scope of the claimed invention. Moreover, there are many uses and applications for this invention in minimally invasive surgery in addition to those noted above.

1. A hooked rod delivery system for use in minimally invasive surgery, which comprises: a substantially cylindrical hollow sleeve defining a bore and having a distal end and a proximal end; and a hooked deployment rod adapted to slide within the hollow sleeve and having a distal end and a proximal end, wherein the distal end of the hooked deployment rod has a hook-shaped member either formed therein or attached thereto.

2. The hooked rod delivery system of claim 1, which further comprises means for holding the hooked deployment rod within the hollow sleeve in either an extended or retracted position.

3. The hooked rod delivery system of claim 2, wherein the means for holding the hooked deployment rod within the hollow sleeve is selected from the group of mechanical holding devices, magnetic holding devices, and combinations thereof.

4. The hooked rod delivery system of claim 3, wherein the means for holding the hooked deployment rod within the hollow sleeve is a spring-loaded mechanical holding device.

5. The hooked rod delivery system of claim 1, wherein the hollow sleeve is prepared from a substantially transparent polymeric material.

6. The hooked rod delivery system of claim 5, wherein a scale or series of gradation marks are contained on an outer surface of the hollow sleeve.

7. The hooked rod delivery system of claim 1, wherein a handle or grip is contained on the proximal end of the hollow sleeve.

8. The hooked rod delivery system of claim 1, wherein a handle or grip is contained on the proximal end of the hooked deployment rod.

9. A hooked rod delivery system for use in minimally invasive surgery, which comprises: a substantially cylindrical hollow sleeve defining a bore and having a distal end and a proximal end; a hooked deployment rod adapted to slide within the hollow sleeve and having a distal end and a proximal end; and means for holding the hooked deployment rod within the hollow sleeve in either an extended or retracted position, wherein the distal end of the hooked deployment rod has a hook-shaped member either formed therein or attached thereto.

10. A hooked rod delivery system for delivering self-closing surgical devices in un-released or un-fired states to operating or surgical sites, which comprises: a substantially cylindrical hollow sleeve defining a bore and having a distal end and a proximal end; and a hooked deployment rod adapted to slide within the hollow sleeve and having a distal end and a proximal end, wherein the distal end of the hooked deployment rod has a hook-shaped member either formed therein or attached thereto.

11. A method for protecting and passing anastomotic devices through laparoscopic and thoracoscopic parts and for delivering such devices to operating or surgical sites, said method comprising:
(a) providing a hooked rod delivery system having a distal end and a proximal end, which comprises: a substantially cylindrical hollow sleeve; and a hooked deployment rod slidably arranged within the hollow sleeve, wherein the hooked deployment rod has a distal end and a proximal end, and wherein the distal end of the hooked deployment rod has a hook-shaped member either formed therein or attached thereto;
(b) causing the distal end of the deployment rod with hook-shaped member to emerge from the hollow sleeve;
(c) engaging an anastomotic device with the hook-shaped member of the deployment rod;
(d) withdrawing the distal end of the deployment rod with engaged anastomotic device into the hollow sleeve of the hooked rod delivery system, thereby compressing the anastomotic device;
(e) positioning the hooked rod delivery system at or near an operating or surgical site by inserting the distal end of the delivery system through laparoscopic or thoracoscopic parts;
(f) moving the deployment rod down toward the operating or surgical site, thereby causing the distal end of the deployment rod with engaged anastomotic device to emerge from the hollow sleeve; and
(g) rotating the deployment rod until the anastomotic device is released or disengaged from the hook-shaped member.

12. The method of claim 11, wherein the anastomotic device is selected from the group of self-closing surgical devices, suture thread, and needles secured to suture thread.

13. The method of claim 12, wherein the anastomotic device is a self-closing surgical device.

14. The method of claim 13, wherein the self-closing surgical device is a self-closing surgical clip assembly that comprises: a flexible thread-like member having opposing first and second ends; a needle attached to the first end of the flexible thread-like member; and a self-closing surgical clip attached to the second end of the flexible thread-like member, and wherein the method further comprises: straightening the needle of the self-closing surgical clip assembly prior to engaging the clip assembly with the hook-shaped member of the deployment rod.

15. A method for delivering self-closing surgical devices in un-released or un-fired states to operating or surgical sites, which comprises: using a hooked rod delivery system that comprises a substantially cylindrical hollow sleeve, and a hooked deployment rod slidably arranged within the hollow sleeve, to protect and pass these self-closing surgical devices through laparoscopic and thoracoscopic parts and to deliver these devices to operating or surgical sites.

16. The method of claim 15, wherein the self-closing surgical device is a self-closing surgical clip assembly.
